(19) United States
(12) Patent Application Publication
(10) Pub. No.: US 2016/0074678 A1
(43) Pub. Date: Mar. 17, 2016

(54) PLANNING SYSTEM FOR TARGETING TISSUE STRUCTURES WITH ULTRASOUND

(71) Applicant: THE TRUSTEES OF COLUMBIA UNIVERSITY IN THE CITY OF NEW YORK, New York, NY (US)

(72) Inventors: Elisa E. Konofagou, New York, NY (US); Thomas Deffleux, Paris (FR)

(73) Assignee: THE TRUSTEES OF COLUMBIA UNIVERSITY IN THE CITY OF NEW YORK, New York, NY (US)

(21) Appl. No.: 14/949,000

(22) Filed: Nov. 23, 2015

Related U.S. Application Data
(63) Continuation of application No. 13/529,239, filed on Jun. 21, 2012, now abandoned.
Application No. 13/529,239 is a continuation of application No. PCT/US2010/061742, filed on Dec. 22, 2010.
(60) Provisional application No. 61/289,299, filed on Dec. 22, 2009.

(51) Int. Cl. A61N 7/00; A61N 7/02; A61B 6/03

(52) U.S. Cl. CPC A61N 7/00; A61B 6/032; A61N 7/02; A61M 2205/3303; A61M 2210/0693; A61M 2205/52; A61N 2007/0052; A61N 2007/0043; A61N 2007/0039

(57) ABSTRACT

The disclosed subject matter provides systems and methods for targeting tissue structures and applying ultrasound thereto. A method according to the disclosed subject matter for targeting a tissue structure using corresponding tissue structure image data includes inputting the tissue structure image data into a targeting simulator, determining acoustic properties of the tissue structure from the corresponding tissue structure image data, and utilizing the determined acoustic properties to align a simulated transducer with the tissue structure such that the tissue structure is targeted. The method can further include acquiring the tissue structure image data, aligning the image data with an atlas of a body structure encompassing the tissue structure and/or selecting parameters of the simulated transducer such that a focal region of an ultrasound wave generated by the simulated transducer targets the tissue structure.

### CROSS REFERENCE TO RELATED APPLICATIONS

This application is a continuation of U.S. patent application Ser. No. 13/529,239, filed Jun. 21, 2012, which is a continuation of International Patent Application Serial No. PCT/US2010/061742 filed Dec. 22, 2010, which claims priority to U.S. Provisional Application No. 61/289,299 entitled “A Planning System for Targeting Specific Brain Structures for Transcranial Blood Brain Barrier Opening,” filed on Dec. 22, 2009, the disclosure of each of which is hereby incorporated by reference herein in its entireties.

### TECHNICAL FIELD

The present application relates to systems and methods for opening a tissue utilizing acoustic parameters in conjunction with microbubbles.

### BACKGROUND

Microbubble-enhanced, transcranial focused ultrasound (FUS) is a highly promising, noninvasive technique shown to open the blood-brain-barrier (BBB) noninvasively, transiently and locally. By opening the blood-brain barrier, larger compounds (>400 Da) that would not otherwise penetrate the brain tissue otherwise can be delivered. The targeted delivery of potential therapeutic agents in small animals has generated renewed interest in the delivery of new drugs in the treatment of neurodegenerative disease in humans. For example, Alzheimer's and Parkinson's treatment stand to benefit significantly from this new delivery technique for promising therapeutic agents such as neurotrophic factors (>15 kDa) or adenoviruses in gene therapy.

On the other hand, scaling from small to large animals can be a daunting task as the ultrasound beam is greatly affected by the skull thickness. Phase aberrations due to discrepancies in sound velocity as well as high absorption can rapidly yield poor focusing and higher energy loss, especially at higher ultrasound frequencies.

Researchers have worked on ways to overcome these effects and obtain a uniform focus through the skull. Many studies were designed for High Intensity Focused Ultrasound (HIFU) therapy, a promising technique used in noninvasive tumor ablation in the brain. HIFU therapy relies on thermal effects, which are dependent on the beam intensity whereas FUS-induced BBB opening relies mostly on mechanical effects such as cavitation (be it stable or inertial), which is linked to the beam pressure and is thus inherently less concentrated than thermal effects.

Transcranial HIFU research has led to the development of complex but very efficient techniques based on multi-element arrays and phase correction techniques relying on prior knowledge of the skull geometry. Using these techniques, higher accuracy and a smaller focus compared to conventional focusing techniques can be achieved, two important conditions given the destructive nature of transcranial HIFU.

On the other hand, sonothrombolysis studies, which use ultrasound to dissolve clots in the brain, generally use lower frequencies which are less prone to phase aberrations and absorption but enhance cavitational effects. The beam is generally loosely focused to cover a large volume of the brain in each application. However, one of these studies led to large, secondary hemorrhage, which has been hypothesized to be linked to unexpected enhanced cavitation effects due to standing waves generated within the skull. Standing waves are known to be capable of trapping microbubbles in antinodes and decrease their inertial cavitation threshold.

### SUMMARY

The disclosed subject matter provides systems and methods for targeting tissue structures and applying ultrasound thereto. A method according to the disclosed subject matter for targeting a tissue structure using corresponding tissue structure image data includes receiving the tissue structure image data into a targeting simulator, determining acoustic properties of the tissue structure from the corresponding tissue structure image data, and utilizing the determined acoustic properties to align a simulated transducer with the tissue structure such that the tissue structure is targeted. The method can further include acquiring the tissue structure image data, aligning the image data with an atlas of a body structure encompassing the tissue structure and/or selecting parameters of the simulated transducer such that a focal region of an ultrasound wave generated by the simulated transducer targets the tissue structure. In some embodiments, the simulated transducer generates an ultrasound wave, and the method further includes calculating standing wave properties of the ultrasound wave in proximity to the tissue structure. In some embodiments, the image data can include acquiring a CT scan and/or a MRI of at least the tissue structure, and determining the acoustic properties of the tissue structure can include determining a pressure waveform of an ultrasound wave moving from the simulated transducer to the tissue structure.

The disclosed subject matter further provides a method for applying ultrasound to a tissue structure which includes receiving the tissue structure image data into a targeting simulator, determining acoustic properties of the tissue structure from the corresponding tissue structure image data, utilizing the determined acoustic properties to align a simulated transducer with the tissue structure such that the tissue structure is targeted, utilizing the alignment of the simulated transducer to align a real transducer with the tissue structure, and applying ultrasound to the tissue structure using the real transducer. The method can further include monitoring the application of ultrasound to the tissue structure, and applying the ultrasound can involve utilizing transducer parameters effective to open the tissue structure. In some embodiments, applying the ultrasound can involve applying ultrasound utilizing transducer parameters effective to disrupt formation of standing waves in proximity to the tissue structure. In the same or other embodiments, the tissue structure can include a brain structure and opening the tissue structure includes opening a blood-brain barrier.

A system for targeting a tissue structure according to the disclosed subject matter includes a targeting simulator comprising a processing unit operatively connected to a memory unit and an input unit, wherein the memory unit contains program instructions operable, when executed by the processing unit, to receive the tissue structure image data, determine acoustic properties of the tissue structure from the corresponding tissue structure image data, and utilize the determined acoustic properties to align a simulated transducer with the tissue structure such that the tissue structure is targeted. In some embodiments, the program instructions are further operable to align the tissue structure image data with an atlas of a body structure encompassing the tissue structure, receive parameters of the simulated transducer selected such that a focal region of an ultrasound wave generated by the simulated transducer targets the tissue structure, and calculate standing wave properties of the ultrasound wave in proximity to the tissue structure. The system can further includes image acquisition devices for acquiring the tissue structure image data.

A system for applying ultrasound to a tissue structure is also provided. The system includes an ultrasound transducer and a targeting simulator. In some embodiments, the program instructions of the targeting simulator are further operable to monitor an application of the ultrasound to the tissue structure.

### BRIEF DESCRIPTION OF THE DRAWINGS

### Color Drawings

The patent or application file contains at least one drawing executed in color. Copies of this patent or patent application publication with color drawing(s) will be provided by the Office upon request and payment of the necessary fee.

The accompanying drawings, which are incorporated and constitute part of this disclosure, illustrate some embodiments of the disclosed subject matter.

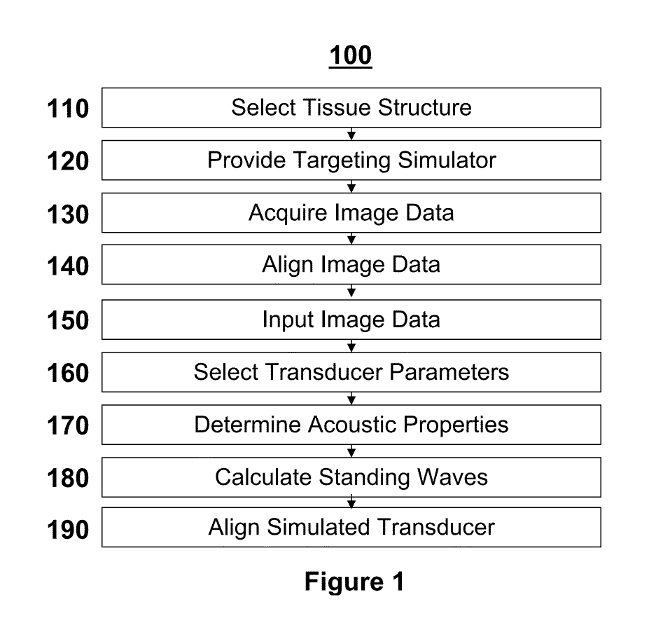

FIG. 1 illustrates a method for targeting a tissue structure in accordance with an exemplary embodiment of the disclosed subject matter.

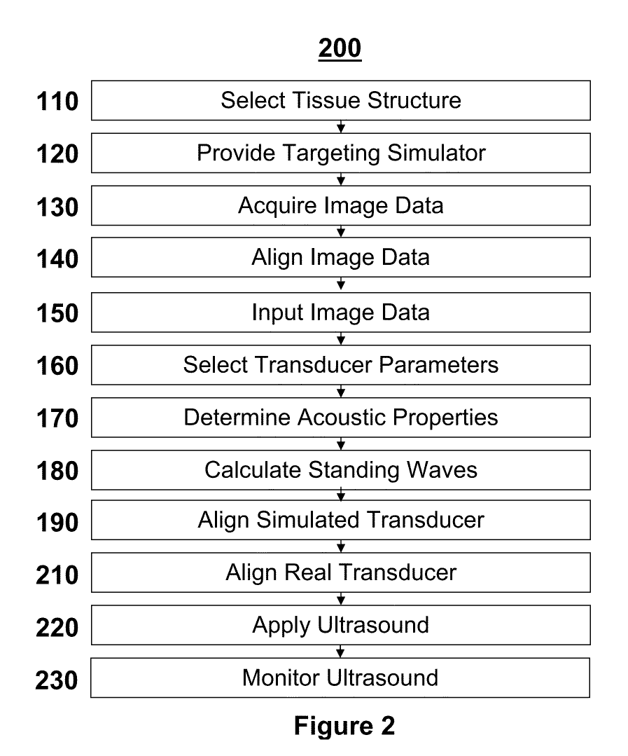

FIG. 2 illustrates a method for applying ultrasound to a tissue structure in accordance with an exemplary embodiment of the disclosed subject matter.

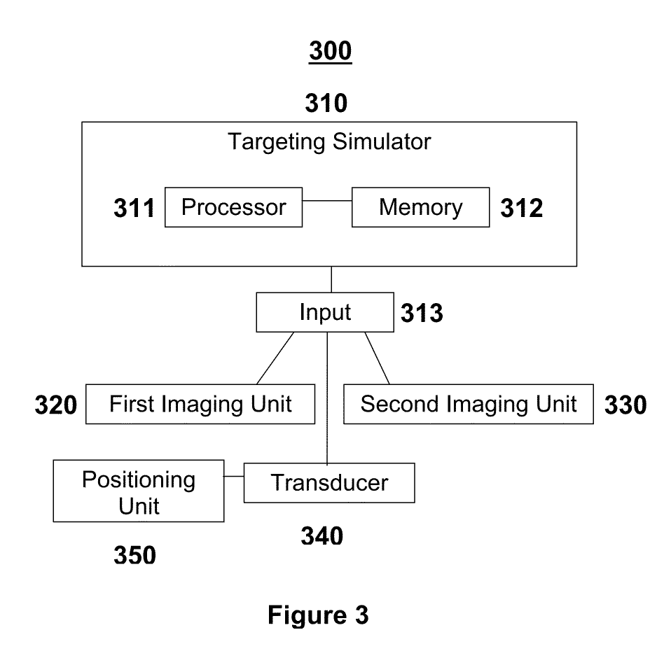

FIG. 3 illustrates a system for targeting a tissue structure and applying ultrasound thereto in accordance with an exemplary embodiment of the disclosed subject matter.

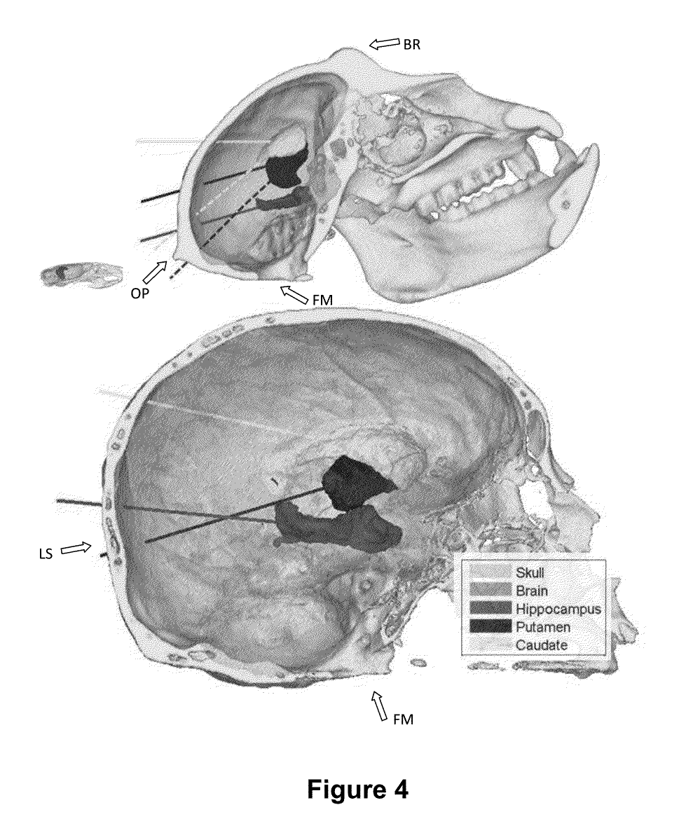

FIG. 4 illustrates mouse, primate and human skull and brain structures, and shows representations of the three targeted tissue structures in accordance with an exemplary embodiment of the disclosed subject matter.

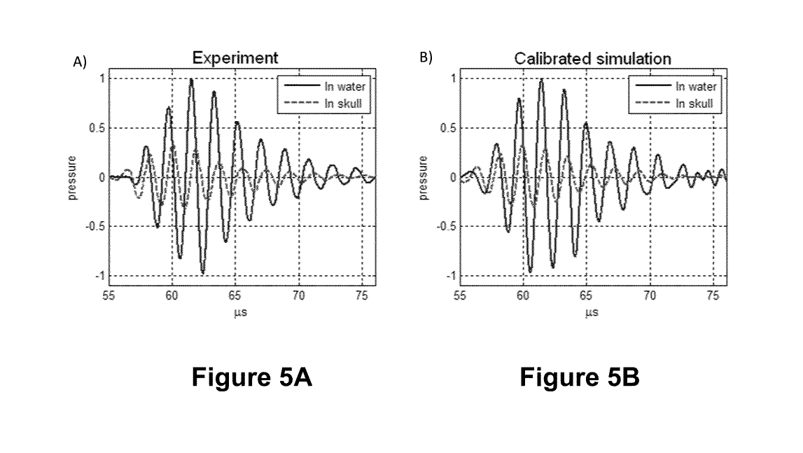

FIG. 5A illustrates a pressure waveform in an in vitro experiment in accordance with an exemplary embodiment of the disclosed subject matter.

FIG. 5B illustrates a pressure waveform determined by a targeting simulator in accordance with an exemplary embodiment of the disclosed subject matter.

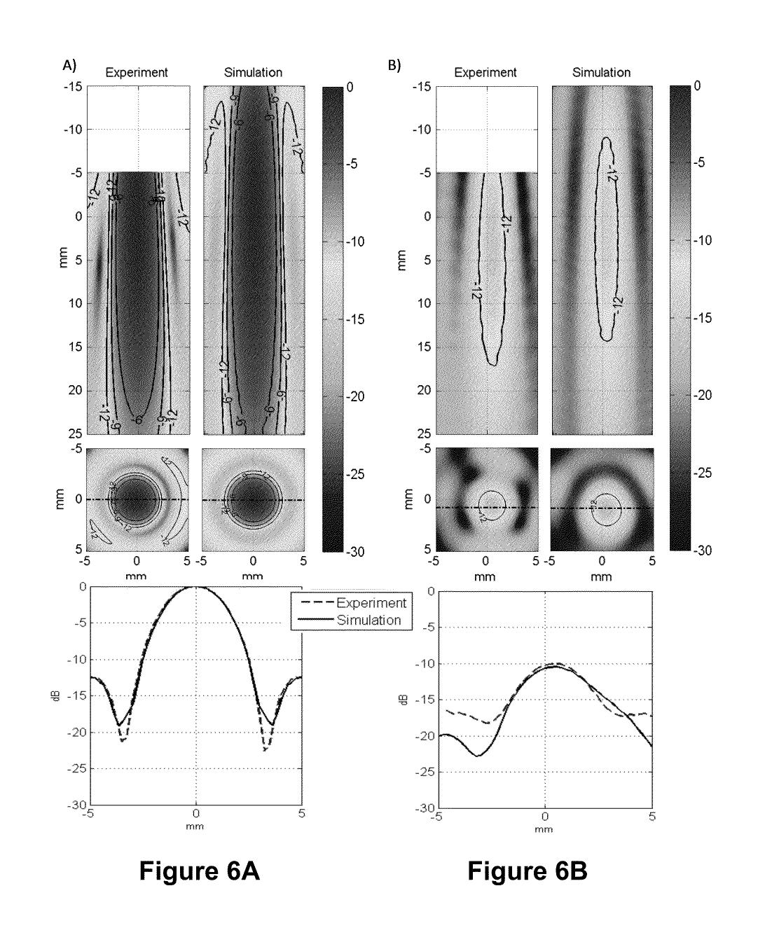

FIGS. 6A-6B illustrate a comparison of experimental and simulated pressure scans in dB for the human skull at 550 kHz in accordance with an exemplary embodiment of the disclosed subject matter.

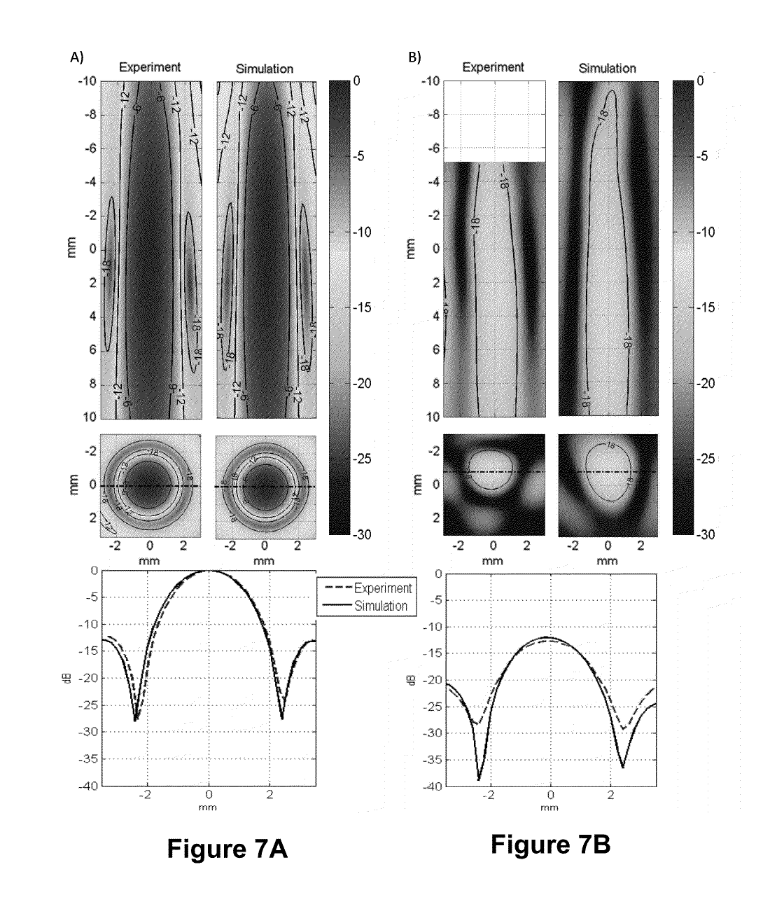

FIGS. 7A-7B illustrate a comparison of experimental and simulated pressure scans in dB for the primate skull at 800 kHz in accordance with an exemplary embodiment of the disclosed subject matter.

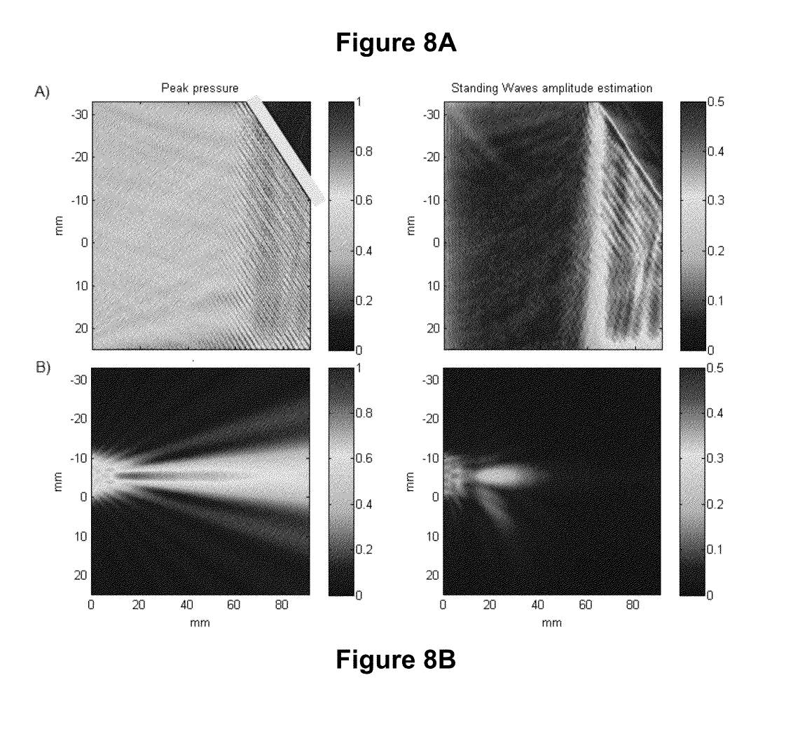

FIGS. 8A-8B illustrate calculation of standing waves in accordance with an exemplary embodiment of the disclosed subject matter.

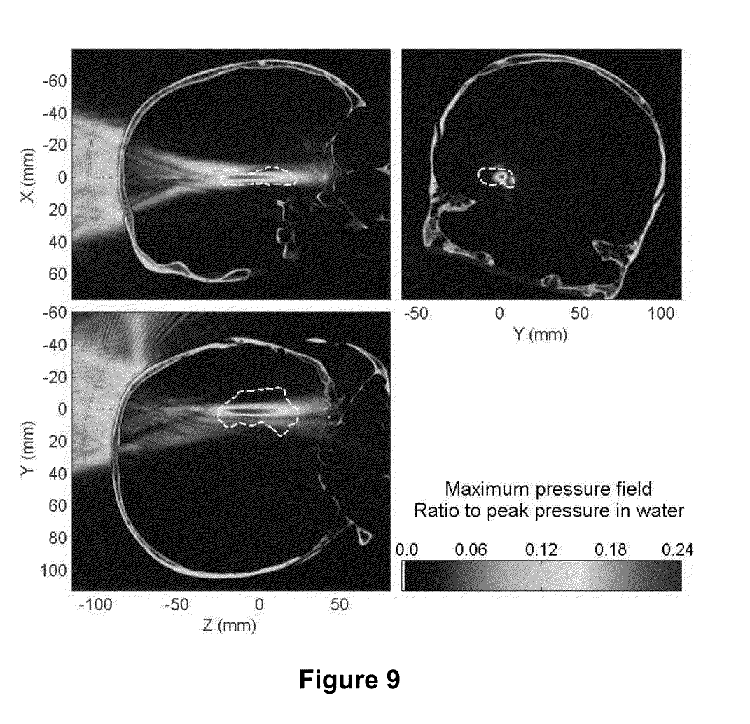

FIG. 9 illustrates the maximum pressure field while targeting the hippocampus through the human skull using a linear chirp at 450-550 kHz in accordance with an exemplary embodiment of the disclosed subject matter.

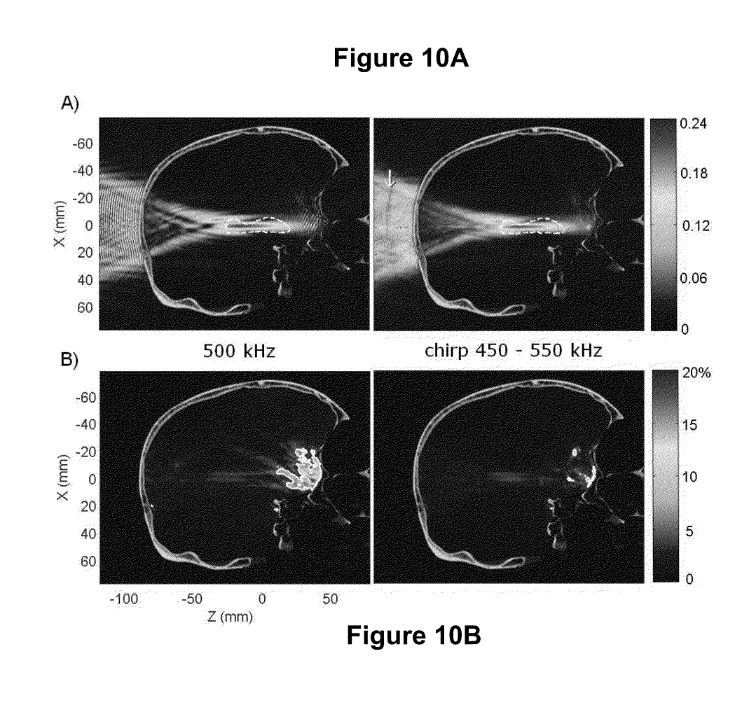

FIG. 10A illustrates pressure fields with a 500 kHz monochromatic beam and with a 450-550 kHz linear chirp in accordance with an exemplary embodiment of the disclosed subject matter.

FIG. 10B illustrates the standing wave amplitude with a 500 kHz monochromatic beam and with a 450-550 kHz linear chirp in accordance with an exemplary embodiment of the disclosed subject matter.

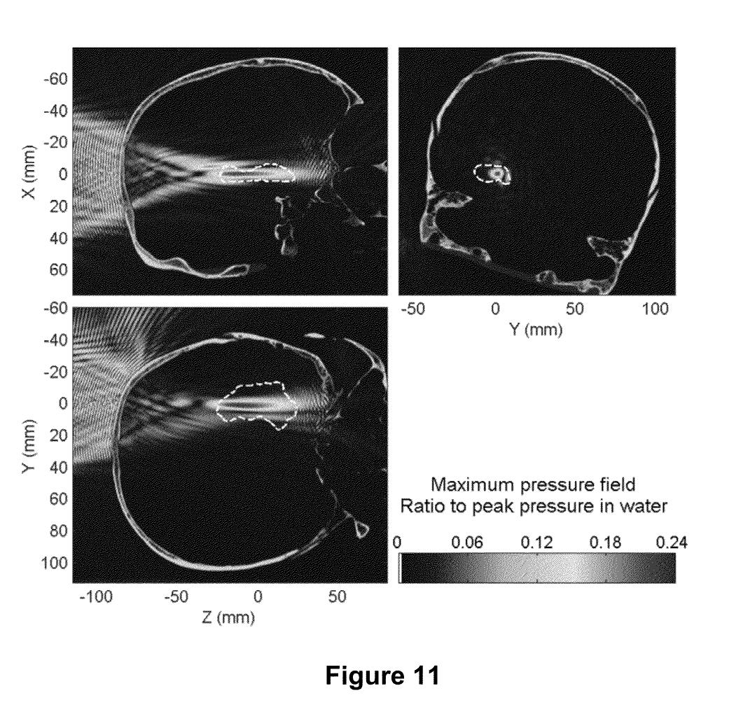

FIG. 11 illustrates the maximum pressure field at 500 kHz while targeting the hippocampus through a human skull in accordance with an exemplary embodiment of the disclosed subject matter.

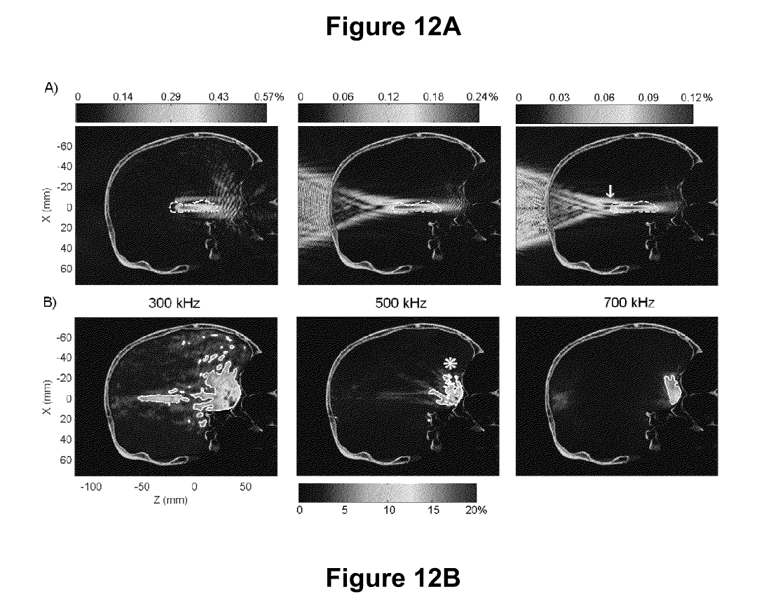

FIG. 12A illustrates pressure fields at 300, 500 and 700 kHz while targeting the hippocampus through a human skull in accordance with an exemplary embodiment of the disclosed subject matter.

FIG. 12B illustrates the corresponding standing wave amplitudes for the same frequencies shown in FIG. 12A in accordance with an exemplary embodiment of the disclosed subject matter.

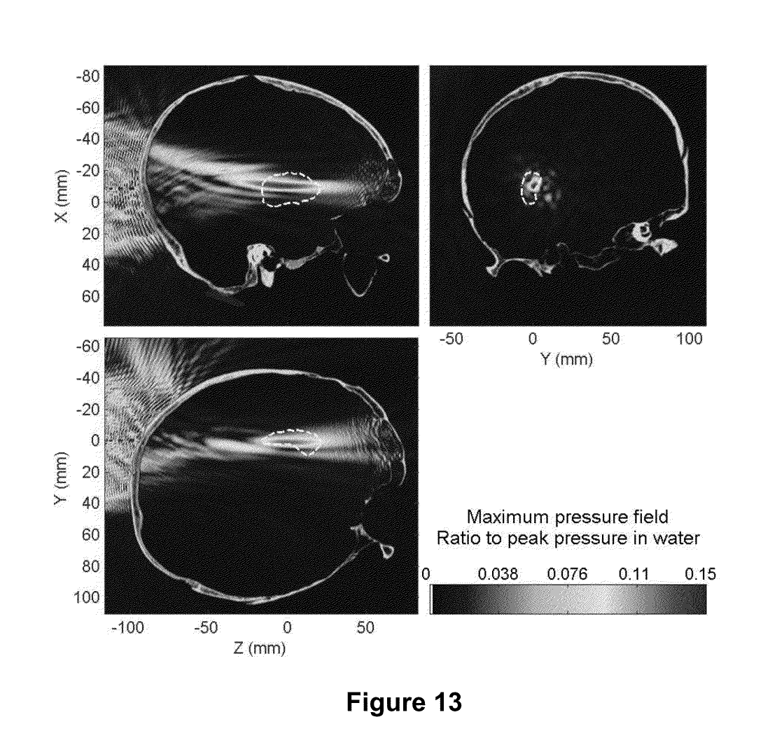

FIG. 13 illustrates the maximum pressure field at 500 kHz while targeting the putamen through the human skull in accordance with an exemplary embodiment of the disclosed subject matter.

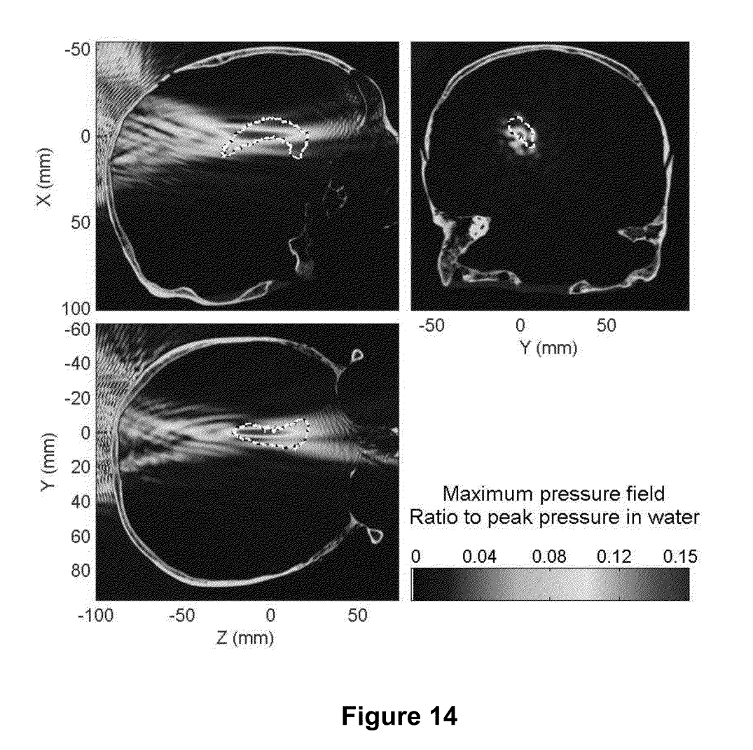

FIG. 14 illustrates the maximum pressure field at 500 kHz while targeting the caudate through the human skull in accordance with an exemplary embodiment of the disclosed subject matter.

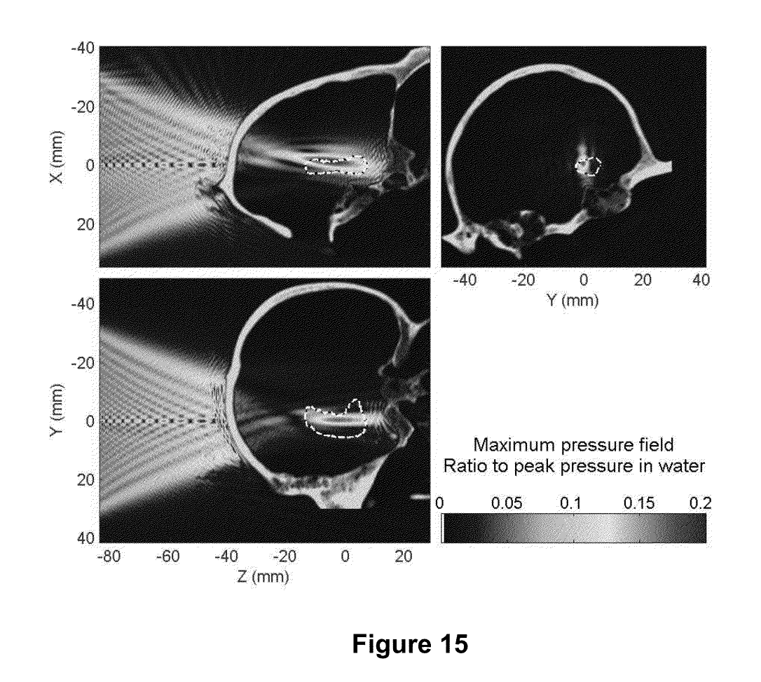

FIG. 15 illustrates the maximum pressure field at 800 kHz obtained with targeting the hippocampus through the primate skull in accordance with an exemplary embodiment of the disclosed subject matter.

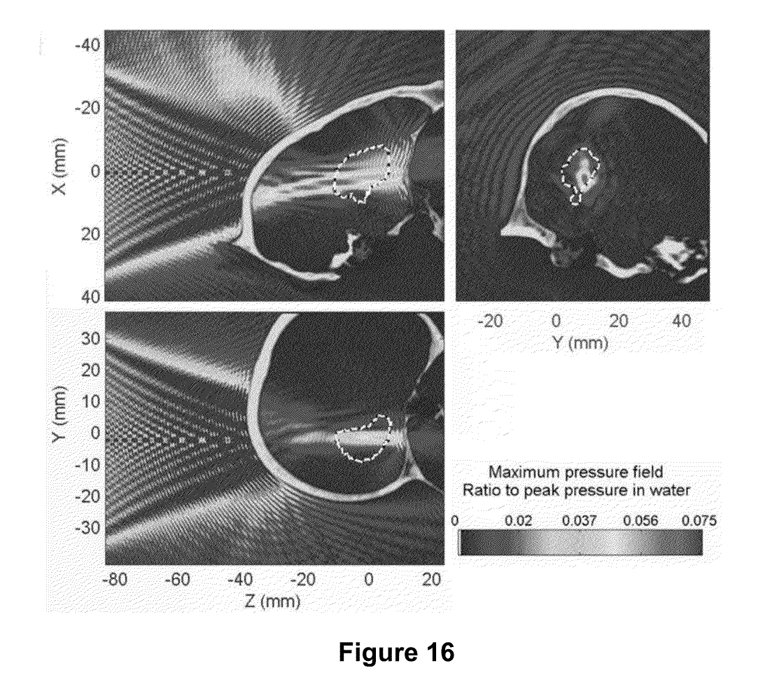

FIG. 16 illustrates the maximum pressure field at 800 kHz obtained with targeting the putamen through the primate skull in accordance with an exemplary embodiment of the disclosed subject matter.

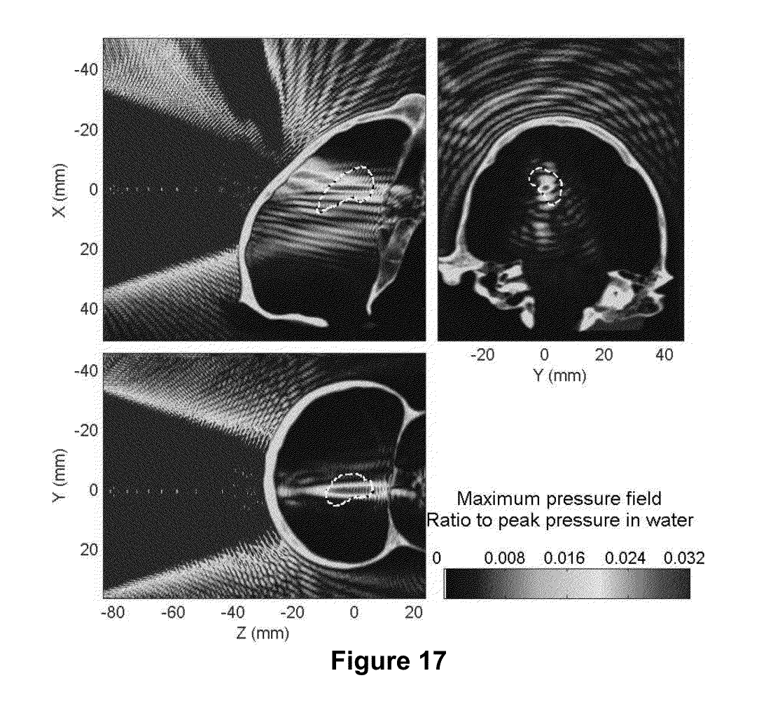

FIG. 17 illustrates the maximum pressure field at 800 kHz obtained with targeting the caudate through the primate skull in accordance with an exemplary embodiment of the disclosed subject matter.

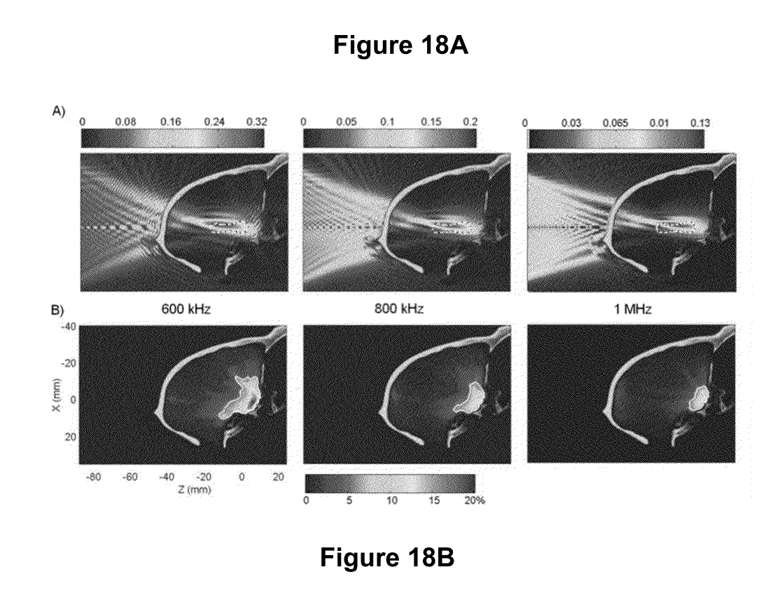

FIGS. 18A-18B illustrate the standing wave amplitude at 600, 800 and 1000 kHz with targeting the hippocampus through the primate skull in accordance with an exemplary embodiment of the disclosed subject matter.

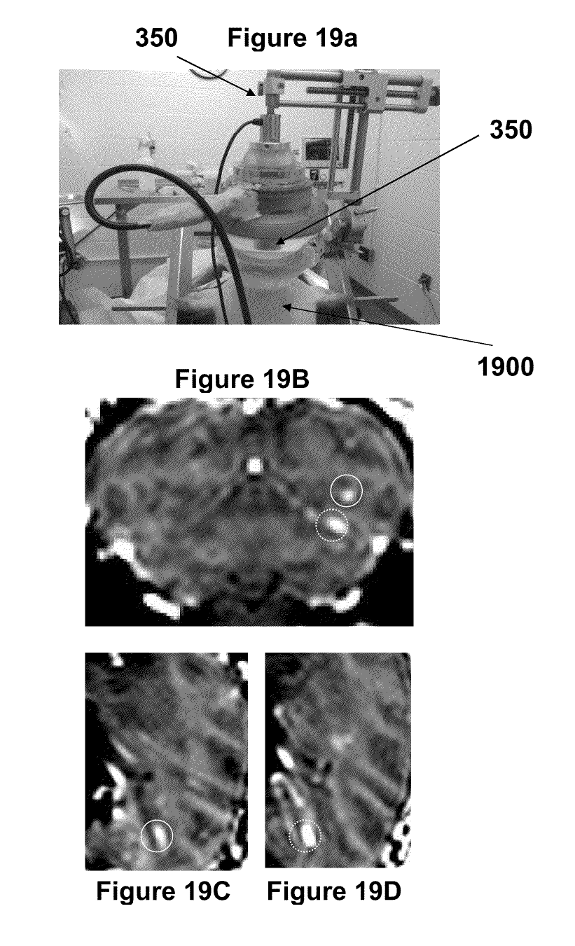

FIG. 19A illustrates a system used in an exemplary embodiment to perform in vivo BBB opening in an anesthetized live monkey in accordance with an exemplary embodiment of the disclosed subject matter.

FIG. 19B illustrates the regions of BBB opening detected on the same coronal plane using the system illustrated in FIG. 19A in accordance with an exemplary embodiment of the disclosed subject matter.

FIGS. 19C-D illustrate two separate sagittal planes showing the BBB opening produced using the system illustrated in FIG. 19A in accordance with an exemplary embodiment of the disclosed subject matter.

### DETAILED DESCRIPTION

The disclosed subject matter provides systems and methods for targeting specific tissue structures of mammals, e.g., non-human primates and humans, for example to target and open the blood brain barrier (BBB). Image data, e.g., an MRI and a CT scan, are received into a targeting simulator which determines the acoustic properties, e.g., the waveform of a simulated pressure wave from a simulated transducer, and aligns the simulated transducer such that the focal region of the simulated transducer targets the desired tissue structure.

The acoustic properties of the simulated transducer are determined using input transducer parameters which can be selected based on the design of a real transducer that will be used on the tissue structure, e.g., a singe spherical transducer operating a low frequencies. Thus the disclosed subject matter can make use of the focalization properties of single transducers at low frequencies (e.g., about 300-1000 kHz) through primate and human skulls to target transcranial structures involved in ultrasound-induced blood-brain barrier opening, such as the hippocampus and the basal ganglia, which are typically affected by early Alzheimer's and Parkinson's disease, respectively.

Unless otherwise defined, all technical and scientific terms used herein have the same meanings as commonly understood by one of ordinary skill in the art to which the disclosed subject matter belongs. Although methods and materials similar or equivalent to those described herein can be used in its practice, suitable methods and materials are described below.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to +/−20%, preferably up to +/−10%, more preferably up to +/−5%, and more preferably still up to +/−1% of a given value. Alternatively, the term can mean within an order of magnitude, preferably within 5-fold, and more preferably within 2-fold, of a value.

The disclosed subject matter illustrates that focusing through the skull with a single spherical transducer at relatively low frequencies (about 300-1000 kHz) can be utilized for BBB opening using a well-formed focal spot obtained in targeted tissue structures. Aberrations of the skull can generate a displacement of the pressure peak, which can be under 2 mm laterally and around 1 cm along the beam axis. The displacement increases with frequency, as aberrations effects are higher, and the skull can appear thicker compared to the wavelength.

Using a threshold for the half-pressure, it is possible to determine the targeting efficiency using the Percent-of-Target-Reached parameter which indicates the percent volume of the targeted structure reached by the beam. The Percent-of-Target-Reached parameter is comparable to what it would be without the skull, which shows that the targeting is minimally affected by the presence of the skull at low frequencies, e.g., 300-1000 kHz. The transducer design utilized herein covered about 11% and 30% of the targeted volume with a single sonication. The entire targeted volume can be covered by mechanically moving the transducer to cover the remained when diffusion mechanism are insufficient.

The Percent-of-Beam-Overlapping-Target parameter indicates the proportion of the field above the half-pressure threshold that is in the targeted structure. It can be approximately 75%, and comparable to the embodiments without the presence of a skull, indicating that most of the BBB opening can occur within the targeted structure. In an embodiment involving a primate skull where the beam path is proximal to the occipital protuberance (for the hippocampus) or to the brow ridge (for the putamen), the Percent-of-Beam-Overlapping-Target parameter can be decreased.

Attenuation has been determined to vary between about 65% and 85% compared to water depending on the geometry and the frequency. For the same skull and frequency, the attenuation can vary greatly with the tissue structure targeted depending to the geometry of the skull in the beam path, affecting the BBB opening consistency.

Accordingly, it has been found that suitable beam orientations can be implemented to avoid higher incident angles and specific bone structures known to cause higher aberration, such as the occipital protuberance in the primate. The beam axis orientation can also take into account the orientations and shapes of the targeted tissue structures. Limiting geometrical effects of the skull in BBB applications can reduce the attenuation and variation of the beam. The beam dimensions can be selected to maximize the overlap with the targeted structures, for example the lateral overlap. In embodiments where the overlap does not cover the entire structure, two or three successive sonications can be used to cover the targeted volume by mechanically moving the transducer. Additionally, defocusing approaches such as the off-axis rotation of the transducer or the use of a toroid-shaped transducer can be used to widen the focal region.

As shown in the disclosed subject matter, the standing wave effect can be calculated based on the analysis of the interferences pattern on the pressure field was implemented. In some embodiments, the maximum amplitude of the standing waves was under 20% of the peak pressure through the skull. Using a threshold of 5% of the maximum peak pressure through the skull, it is possible to calculate the extent of the standing waves. The region where the standing wave effect can be significant was small, e.g., under 2% of the brain volume, and decreased with frequency, as the tissue absorption increased. Large standing-wave amplitudes during long application times can increase the risk of hemorrhages as illustrated by the TRUMBI sonothrombolysis study with total cumulated active sonication duration of more than 4 min at 300 kHz.

As shown in the disclosed subject matter, fast linear chirps can be used to reduce the standing wave amplitude. Interferences are not localized when chirps are used and the brain volume where standing wave amplitude is significant (higher than 5% of the peak pressure through skull) is divided by 5. The maximum standing wave amplitude decreased from 19% to 12% due to the localization of the maximum close to the skull interface where the chirp is less efficient since the frequency difference between the reflected and incident waves is small. The technique allows physical effects taking longer than 20 μs to occur to see a uniform pressure field as nodes and antinodes will be smoothed over this timescale.

The disclosed subject matter also provides a method for utilizing the targeting simulator in a BBB opening application using, e.g., a single-element transducer. Tabulated values for the coordinates and orientation of tissue structures based on an average over a population can be input into the simulation. Corrections can also be applied based on the patient's actual head size to improve accuracy. Further, true automatic segmentation of the targeted structures using prior MRI data of the patient can be used to find the tissue structures and align the transducer for each case. Since MRI is performed in the case of Alzheimer's and Parkinson's diagnosis and monitoring, such data are already available. Corrections to the targeting parameters, e.g., the focus displacement, such as the skull lens effect, can also be implemented based on the MRI data. The simulation results can be used, along with a stereotactic frame, to position and align the transducer with the structure main axis.

As shown in the disclosed subject matter, the simulation can be utilizing for real time monitoring of the sonication. For example, if the internal pressure cannot be accurately predicted, a progressive increase of the pressure can be used together with a monitoring technique such as passive cavitation detection or gadolinium imaging by MRI. The system described herein can use any transducer, focused or unfocused, spherical or non-spherical, as long as a focal spot can be formed. The transducer can be selected with smaller or larger focal spot, or moved laterally to increase the targeted volume and include the entire region or regions targeted depending on the extent of the disease.

FIG. 1 illustrates a method 100 for targeting tissue structures using corresponding tissue structure image data in accordance with the disclosed subject matter. Method 100 can include selecting 110 a tissue structure of interest and providing 120 a targeting simulator, which in some embodiments can be a finite-difference, time-difference simulation platform capable of solving a three-dimensional linear, acoustic wave equation. Method 100 can also include acquiring 130 tissue structure image data, e.g., an MRI and/or a CT scan of the tissue structure, and receiving 150 the tissue structure image data into the simulator. The method 100 can include aligning 140 the MRI and CT scans with an atlas of the body structure surrounding the tissue structure of interest. In the case where the tissue structure of interest is a brain structure, the MRI and/or CT scans can be aligned 140 with an atlas of the skull.

Method 100 can also include selecting 160 transducer parameters, for example, the focal length and frequency of operation, such that a focal region of an ultrasound wave generated by the simulated transducer targets the tissue structure. Method 100 further includes determining 170 acoustic properties of the tissue structure of interest and, in some embodiments, the encompassing body structure, e.g., the surrounding brain matter and the skull in embodiments involving the brain. In some embodiments, the acoustic properties are determined 170 across the three-dimensional space from the position of a simulated transducer to the tissue structure that is being targeted. In one embodiment, the acoustic properties determined 170 can include, for example, the acoustic density, the velocity of sound, the attenuation, the correlation length and the pressure waveform of an ultrasound wave. The pressure waveform can be determined 170 utilizing the other determined properties, e.g., the acoustic density, the velocity of sound, the attenuation, the correlation length, the bulk viscosity, the shear viscosity and/or the vector displacement, of an ultrasound wave moving through various tissues, e.g., skull and brain tissue, to reach the tissue structure of interest. The pressure waveform can also be determined 170 utilizing the selected 160 transducer parameters such as, for example, the frequency and focal length. The pressure waveform can be determined 170 over the 3D volume of material, e.g., skull and brain matter in the case of a BBB opening embodiment, between the transducer and the tissue structure which is being targeted. Method 100 can also include calculating 180 standing wave properties, utilizing, for example, a high spatial filter on the maximum pressure field to isolate spatial modulation due to constructive and destructive interferences of the waves.

Method 100 further includes utilizing the determined 160 acoustic properties to align 190 the simulated transducer with the tissue structure of interest such that it is targeted. In some embodiments, the simulated transducer is aligned 190 to be co-axial with the longest dimension of the tissue structure. In this manner the alignment 190 can be effective to maximize area of the tissue structure covered by the transducer focal spot. In an embodiment utilizing the method 100 in connection with BBB opening, the alignment 190 of the transducer is selected to maximize the area of the BBB opened with each ultrasound shot.

FIG. 2 illustrates a method 200 for targeting and sonicating a tissue structure. Method 200 can include all the elements of method 100, as detailed above. Method 200 further includes aligning 210 a real transducer with the tissue structure of interest, and applying 220 ultrasound to that tissue structure. In some embodiment, applying 220 ultrasound can include applying ultrasound effective to open the targeted tissue structure, such as the BBB. Systems and methods for opening the BBB are disclosed in commonly assigned U.S. Patent Publication 2009/0005711, which is incorporated by reference in its entirety herein. Applying 220 ultrasound can also include selecting 160 and utilizing transducer parameters effective to disrupt formation of standing wave in proximity to the tissue structure. In some embodiment, this involves using a linear chirp waveforms, whereby the ultrasounds waveforms are generated by varying the signal frequency in time. Method 200 can further include monitoring 230 the application of the ultrasound using the targeting simulator to, for example, determine the extent to which the tissue structure of interest is being covered by the ultrasound beam. This can include determining the Percent-of-Target-Reached parameter and/or the Percent-of-Beam-Overlapping-Target parameter.

FIG. 3 illustrates a system 300 for targeting a tissue structure. System 300 includes a targeting simulator 310, which can be on a computer and can include a processing unit 311, a memory unit 312, and can be operatively connected to an input unit 313. In some embodiments system 300 can also include a first imaging unit 320, e.g., a CT imaging unit, and a second imaging unit 330, e.g., a MRI unit, both of which can be operatively connected to the input unit 313 in order to input the image data into the targeting simulator 310. System 300 can also include a transducer 340, which can be, e.g., a single-element transducer and can be operatively connected to the input unit 313 to input data from the transducer into targeting simulator 310. In some embodiments, system 300 can further include a positioning unit 350, e.g., a stereotactic, for positioning the transducer 340.

In one embodiment of the disclosed subject matter, selecting 110 a tissue structure for targeting can include selecting tissue structures which would be clinically relevant to specific medical conditions, such as Alzheimer's and Parkinson's disease. In one embodiment, the hippocampus was selected 110 for its predominant role in Alzheimer's disease. FIG. 4 illustrates mouse, primate and human skull and brain structures, and shows representations of the three targeted tissue structures, the hippocampus and the putamen and the caudate, which are both parts of the basal ganglia. The axes chosen for the orientation of the transducer in the simulation are also represented. The mouse skull is also represented and skulls are to scale. FM denotes the foramen magnum hole, LS the lambdoid suture, OP the occipital protuberance and BR the brow ridge. The dashed lines (for the primate skull only) represent the main axes of the putamen (blue) and the caudate (yellow) that were not used in a simulation since those axes pass through the occipital protuberance.

The striatum, the predominant structure of the basal ganglia, which encompass the putamen and the caudate nucleus, can be selected 110 at least partially due to its role in the dopamine pathway, which is a pathway severely altered by Parkinson's disease. The substantia nigra, a very small structure of the basal ganglia is known to be at the beginning of the dopamine pathway and can also be selected 110 depending on the drug to be used in treatment and the state of the disease. The tissue structures selected 110 can be identified for all species using three-dimensional brain atlases.

In one embodiment, the targeting simulator provided 120 can be a numerical simulator utilizing a linear full-wave 3D finite-difference time-domain (FDTD) commercial package (e.g., Wave 3000, CyberLogic, New York, USA). In this embodiment, the package solves the linear equation:

$$ρ \frac{{\partial }^{2} U}{\partial {t}^{2}} = [ μ + η \frac{\partial }{\partial t} ] {∇}^{2} U + [ λ + μ + φ \frac{\partial }{\partial t} + \frac{η}{3} \frac{\partial }{\partial t} ] ∇ ( ∇ \cdot U ) , \qquad ( Eq . 1 )$$

where ρ is the material density [kg.m⁻³], λ is the first Lame constant [N.m⁻²], μ is the second Lame constant [N.m⁻²], φ is the bulk viscosity [N.s.m^{31 2}], η is the shear viscosity [N.s.m⁻²], U is the 3D vector displacements field [m.s⁻¹]. Equation 1 can be utilized to determine 170 the pressure waveform of an ultrasound wave as it moves through various tissues, such as skull and brains in the case of a BBB opening embodiment.

The linearity of the model can limit the overall computation time of each simulation. The non-linear contribution will be low at the pressure threshold of BBB opening (0.3 MPa at 1.5 MHz). In one embodiment, viscoelasticity was not modeled, e.g., μ(x,y,z) and η(x,y,z) in equation 1 were set to zero, since shear waves do not propagate in liquid media and mode conversion from compressional waves to shear waves inside the skull is not significant for an incidence angle lower than 20°. The simulation was carried out on a 64-bit workstation 310 with 4-dual core 2.3 GHz Xeon processors 311 and 32 GB of RAM 312 (Precision WorkStation 690, Dell, Austin, Tex., USA).

In one embodiment, the tissue structure image data acquired 130 includes a CT image of a skull, e.g., a primate skull. The primate skull used was part of the Macaca Mulatta species, also known as the Rhesus monkey. The formalin-fixed skull was 145 mm long, 85 mm high and 69 mm wide for a thickness of 2.6±0.2 mm and a brain volume of 85 cm³. The foramen hole has a diameter of 15.2±0.45 mm and was used for the in vitro pressure scans as well as the calibration of the acoustic parameters. The full 3D CT scan of the skull was acquired 130 on a GE LightSpeed VCT 64 scanner 320 (GE Medical Systems, Milwaukee, Wis., USA) with a native 488-μm resolution and slice thickness of 625 μm.

In one embodiment where the tissue structure image data acquired 130 included a CT image of a human skull, the human skull was approximately 195 mm long, 145 mm high and 148 mm wide for an average thickness of 5.75±0.72 mm and a brain volume of 1500 cm³. The foramen hole had a diameter of approximately 21±2.2 mm and was also used for the in vitro pressure scan and calibration. The image data was acquired 130 with a GE LightSpeed VCT 64 scanner utilizing the same parameters used for the monkey skull. The scanner was used to acquire 130 a full 3D CT scan leading to an average of 16 samples of the CT density function through the skull thickness. Persons of skill in the art will understand that higher resolution scanners can be used to acquire 130 the image data with finer heterogeneities of the skull.

In some embodiments, the CT scan can be segmented utilizing techniques known in the art, e.g., active contours using level sets, in order to separate the skull from the background (e.g., the noise) and also to extract the brain volume in 3D for alignment 140, e.g., co-registration, with the atlases.

After segmentation of the intra skull volume based on the CT scans, a three-dimensional brain atlas can be aligned 140 with the skull using, e.g., an affine transformation (translation, rotation, scaling and shearing) in Matlab (R2008b, The Mathworks, Inc., Natick, Mass., USA). In one embodiment involving rhesus monkeys, an atlas of the monkey brain was provided by the University of North Carolina. In one embodiment involving humans, a publicly available ICBM (International Consortium for Brain Mapping) template from the Laboratory of Neuro Imaging, UCLA was used as an atlas of the human brain. In other embodiments, acquiring 130 the image data can include acquiring an MRI of the brain, which can be used in place or in conjunction with a pre-existing brain atlas. Accordingly, in such embodiments the CT scan can be aligned 140 with the MRI and/or the pre-existing brain atlas.

Method 100 includes receiving 150 the tissue structure image data into the targeting simulator. The data can be received 150 by any means known in the art, such as by CD, USB, wirelessly, or by utilizing a network to transfer the data in a specific format, e.g., a binary format and a configuration file.

Utilizing the alignment 140 of the image data, e.g., the atlas with the CT scan, acoustic properties including properties of the tissue structure can be determined 170. In one embodiment, the center of mass and long axis of each targeted structure can be determined using principal component analysis (PCA). As detailed below, such features of the tissue structure can be used for aligning 190 the position and orientation of simulated transducer to provide the desired overlap between the expected focal spots and the targeted structures. The rough dimensions of the brain structures in some embodiments are summarized in Table 1.

TABLE 1
Brain Structure Dimensions

| | Mouse | Primate | | | Human | | |
|---|---|---|---|---|---|---|---|
| | Left | Left | Left | Left | Left | Left | Left |
| | Hippocampus | Hippocampus | Putamen | Caudate | Hippocampus | Putamen | Caudate |
| Length | 2.8 mm | 18 mm | 17.5 mm | 19 mm | 46 mm | 35 mm | 43 mm |
| Diameter | 0.9 mm | 3.5 mm | 6 mm | 5.5 mm | 14.5 mm | 12.5 mm | 13 mm |
| Depth | 2.3 mm | 40 mm | 50 mm | 55 mm | 90 mm | 98 mm | 105 mm |

Method 100 can further include selecting 160 the parameters of the simulated transducer such that a focal region of an ultrasound wave generated by the simulated transducer targets the tissue structure. In some embodiments, the parameters can be selected 160 so as to provide a focal spot tailored to the targeted structure dimensions and with a focal length long enough to accommodate the tissue structure dimensions, e.g., the selected brain structure dimensions. The active diameters of the transducers can also be selected 160 for certain frequencies to provide comparable focal spot dimensions between different embodiments, such as embodiments involving monkey brains and embodiments involving human brains. In one embodiment, an estimation of the focal dimensions in water for each design were computed with the Field II simulation software using the conventional −3 dB definition.

In an embodiment involving a human brain, a focal length of 140 mm was selected 160 to accommodate the depth of the selected tissue structures. For a 500-kHz center frequency, a Focal/Diameter number of 1.4 (diameter of 100 mm) can be used since it provides, in water, a focal size of 42 mm long and 4.5 mm wide, comparable with all sizes of the selected brain regions, e.g., the hippocampus, putamen and caudate. Its width is only one third of the average diameter of the structures but it is can be expected that diffusion of smaller compounds inside the extracellular, extravascular space could reach a larger fraction of the targeted structure. In one embodiment involving the delivery of larger compounds with a lower diffusion coefficient, the transducer can be mechanically moved to repeat the sonication at different locations, and thereby to cover the full selected tissue structure. Utilizing such a transducer, the acoustic focus in water can be at an axial distance of 2 mm from the geometric focus closer to the transducer. Using different frequencies can require the use of different diameters each time in order to approximately maintain the same focal spot dimensions. Thus, in one embodiment, a 42-mm diameter transducer was used at a frequency of 700 kHz (having focal spot dimensions: 40×3.4 mm²) and a 128-mm diameter transducer for a frequency of 300 kHz (having focal spot dimensions: 40×5.2 mm²).

In an embodiment involving a primate brain, a transducer with a focal length of 90 mm can be selected 160. A Focal/Diameter number of 1.25 (diameter of 72 mm) was selected 160 at 800 kHz, corresponding to a focal size in water of 2.4 mm×22.2 mm. Similarly, diffusion effects can expand the delivery area of compounds within the selected tissue structure. Frequencies of 600 kHz and 1 MHz correspond to a diameter of 64 mm at 1 MHz (focal spot dimensions: 19.5×2 mm) and a diameter of 80 mm at 600 kHz (20.4×2.6 mm). Values for primate and human embodiments where the hippocampus was selected 110 are summarized in Table 3, with the focal size kept constant at all frequencies by adjusting the diameter of the transducer.

TABLE 2
Transducer parameters.

| | Primate Embodiment | | | Human Embodiment | | |
|---|---|---|---|---|---|---|
| | 600 kHz | 800 kHz | 1 MHz | 300 kHz | 500 kHz | 700 kHz |
| Focal length | 90 mm | 90 mm | 90 mm | 140 mm | 140 mm | 140 mm |
| Diameter | 80 mm | 72 mm | 64 mm | 140 mm | 100 mm | 84 mm |
| Expected focal | 21 × 2.8 mm² | 22 × 2.4 mm² | 22 × 2 mm² | 40 × 5.2 mm² | 42 × 4.5 mm² | 42 × 3.6 mm² |
| spot size | | | | | | |

Method 100 can further include determining 170 he acoustic parameters of the tissue structure from the corresponding tissue structure image data, for targeting tissue structure and its surrounding medium. In embodiments involving the brain, the properties of the brain matter and the skull can be determined 170. The acoustic properties of the skull can be determined 170 utilizing a simple homogenous layer model with thickness assessed by CT scans. In other embodiments, a full 3D heterogeneous map of the skull based on the CT apparent density can be used to determine 170 the properties of the skull.

In this embodiment, the acoustic density p and velocity c maps are assumed to be proportional to the apparent CT density maps. If ρ_{CT} is the normalized apparent CT density (0≦ρ_{CT}≦1), then the acoustic properties of the skull, e.g., the properties inside the skull material, can be determined 170 as follows:

ρ(x,y,z)=ρ₀+(ρₘₐₓ−ρ₀)×ρ_{CT}(x,y,z), (Eq. 2)

c(x,y,z)=c₀+(cₘₐₓ−c₀)×ρ_{CT}(x,y,z), (Eq. 3)

α(x,y,z)=αₛₜₐₗₗ (Eq. 4)

where ρ₀ is the acoustic density of water, ρₘₐₓ is the maximum acoustic density in the skull [kg.m⁻³], c₀ is the water velocity, cₘₐₓ is the maximum velocity in the skull [m.s⁻¹], αₛₖᵤₗₗ is the skull attenuation [dB.m⁻¹]. In some embodiments, the acoustic properties determination 170 can be simplified by assuming the attenuation to be homogeneous inside the skull. In other embodiments, more complex absorption models can be utilized.

The acoustic properties of the medium surrounding the tissue structure, e.g., the skull, can be further determined 170 utilizing CT density maps sampled to an isotropic resolution on the order of, e.g., 100-250 μm, using spline interpolation to preserve boundaries. In embodiments involving human and primate skulls, the CT density maps were sampled at 250 μm for the human skull and 200 μm for the primate skull. Such resolutions can provide the necessary stability for the FDTD algorithm, although the finest heterogeneities can require resolutions on the order of 200 μm. In one embodiment, the time step was automatically adapted by the targeting simulator to satisfy the Courant stability criterion in the absorbing media.

In some embodiments, certain acoustic properties, e.g., brain density, can be determined 170 from pre-existing sources and can be assumed to be similar in human and monkey skulls. In the same or other embodiments, certain acoustic properties, e.g., the attenuation and sound velocity in the skull and brain, can be determined 170 from in vitro or in vivo measurements. Using a series of transient pulses, the time of flight and attenuation can be experimentally measured 170 and compared with the simulation determination 170 of the same transducer, positioning and excitation pulse parameters. Values of skull attenuation of αₛₖᵤₗₗ and maximum sound velocity cₘₐₓ can be modified in equations 3-4 from their initial values to provide a best match between simulation and experimental determinations 170.

FIGS. 5A and 5B illustrate an example of pulses in an embodiment involving a human skull, after the adjustment of the acoustic parameters. FIG. 5A shows the pressure waveform in an in vitro experiment and FIG. 5B shows the pressure waveform determined 170 in by the targeting simulator utilizing the in vitro results to calibrate the maximum sound velocity and absorption of the human skull. The acoustic properties used in one embodiment are provided in Table 3. The values in Table 3 were determined 170 using pre-existing sources, e.g., from Kremkau et al., “Ultrasonic attenuation and propagation speed in normal human brain,” The Journal of the Acoustical Society of America, vol. 70, p. 29, 1981 and Pichardo et al., “Multi frequency characterization of speed of sound for longitudinal transmission on freshly excised human skulls,” in 9th International Society on Therapeutic Ultrasound, 2009, p. 136, each of which is incorporated by reference in its entirety herein. The sound velocity and attenuation were adjusted from utilizing experimental measurements.

TABLE 3
Brain and skull acoustic properties used in an embodiment.

| | | Density (kg/m³) | | Velocity (m/s) | | | |
|---|---|---|---|---|---|---|---|
| | | max | average | max | average | Attenuation | |
| Water | | — | 1000 | — | 1540 | 2.5 | Np/m/MHz |
| Human skull | 300 kHz | ″ | ″ | ″ | ″ | no attenuation | |
| | 500 kHz | ″ | ″ | ″ | ″ | 60 | Np/m |
| | 550 kHz | 2190 | 1895 | 3100 | 2690 | 80 | Np/m |
| | 700 kHz | ″ | ″ | ″ | ″ | 130 | Np/m |
| Primate skull | 600 kHz | ″ | ″ | ″ | ″ | 50 | Np/m |
| | 800 kHz | 2190 | 1900 | 3100 | 2550 | 120 | Np/m |
| | 1 MHz | ″ | ″ | ″ | ″ | 190 | Np/m |
| Human Brain | | — | 1000 | — | 1560 | 10 | Np/m/MHz |
| | | | | | | (5 Np/m at 500 kHz) | |
| Monkey Brain | | — | 1000 | — | 1560 | 10 | Np/m/MHz |

Sound velocity can be assumed to be frequency independent and attenuation can be assumed to follow a linear relationship with frequency in the frequency range used in one embodiment (e.g., 300-1000 kHz). A slope of 350 Np/m/MHz can also be assumed for both a human and primate skull based on homogenized attenuation values found in Connor, W., “Simulation methods and tissue property models for non-invasive transcranial focused ultrasound surgery,” Ph.D. Thesis, Harvard University-MIT Division of Heath Sciences and Technology, 2005, which is incorporated by reference in its entirety herein.

Simulations can be performed at different frequencies to estimate the correlation lengths of both skulls (through the parietal bone). Table 4 illustrates the results which show a drop of the correlation length with the frequency indicating an increase in beam aberration. The correlation lengths in the primate skull are larger than in the human skull partially due to the skull being thinner. The primate correlation lengths were determined through the parietal bone, e.g., without taking into account higher incidence angle and specific bone structures.

TABLE 4
Correlation Lengths

| | 300 kHz | 500 kHz | 600 kHz | 700 kHz | 800 kHz | 900 kHz | 1000 kHz |
|---|---|---|---|---|---|---|---|
| Human skull | 47.5 mm | 27.4 mm | • | 18.2 mm | • | 10.6 mm | • |
| Primate skull | • | • | 34.3 mm | 27.8 mm | 20.3 mm | 17.9 mm | 13.4 mm |

In one embodiment, in vitro measurements were conducted as part of the determination 170 of the acoustic properties, e.g., of a human and primate skull. The measurements were conducted with a 0.2 mm needle hydrophone (Precision Acoustics Ltd., Dorchester, Dorset, UK) and acquired on a computer with an 80-MHz digital acquisition board (model 14200, Gage applied technologies Inc., Lachine, QC, Canada). The hydrophone was suspended from a linear 3D axis positioning system (Velmex Bloomfield, N.Y., USA) and used for raster scanning. Skulls, e.g., human and primate skulls, were soaked into degassed water for several hours prior to all measurements.

In this embodiment, two transducers were used. The first transducer (Imasonic, Tours, France) had a center frequency of 1.3 MHz (active diameter=60 mm, inner hole diameter=16.5 mm, focal distance=60 mm, bandwidth at half-power=43%) and was used for the monkey skull measurements and calibration, using a 800 kHz frequency. The second transducer (Riverside Institute, New York, N.Y., USA) had a lower center frequency of 550 kHz (active diameter=80 mm, inner hole diameter=24 mm, focal distance=90 mm, bandwidth at half-power=15%) and was used for the human skull measurements and calibration at lower frequencies.

For all measurements, the highest pressure peak was first identified in water and a three-dimensional (3D) raster scan was successively performed. The skulls were then carefully positioned with the hydrophone kept centered inside the foramen magnum hole, as illustrated in FIG. 4, and the transducer facing the top of the skull at a controlled distance.

Pressure scans inside the skulls were acquired (30×10×10 mm³ at 550 kHz for the human skull, 15×6×6 mm³ at 800 KHz for the primate skull) and normalized by the peak pressure in water with all parameters kept identical. Additionally, a series of 30 transient pulses (4 cycles) were averaged and recorded at the acoustic focus, first in water and then in the presence of a skull. They were used as described previously for the calibration of sound velocity and attenuation.

FIGS. 6A and 6B illustrate a comparison of experimental and simulated pressure scans in dB for the human skull at 550 kHz. Initial skull positioning errors in the simulation model compared to the experiment can be estimated to be a few millimeters. Pressure scans were deliberately centered below the focus to avoid any possible contact between the hydrophone and the skull during scans, as illustrated by the cut-off portion at the top of FIGS. 6A and 6B.

FIGS. 7A and 7B illustrate a comparison of experimental and simulated pressure scans in dB for the primate skull at 800 kHz. Pressure scans were deliberately centered below the focus point to avoid any possible contact between the hydrophone and the skull during scans, as illustrated by the cut-off portion at the top of FIGS. 7A and 7B.

In some embodiments the accuracy of the targeting simulator can be determined by comparing the pressure field, e.g., the maximum pressure field, between the in vitro pressure scan and the simulated pressure field for the primate and human skulls. As described above, in such in vitro experiments the transducer was positioned above the top of the skull and the hydrophone was inserted through the foramen magnum hole.

In one embodiment, the maximum pressure field was determined 170 using 20 pressure field samples in one period in the assumed pseudo state. The simulation durations were set to 200 μs for the human skull and 150 μs for the monkey skull corresponding to a propagation distance of the ultrasound beam of 30 cm and 24 cm, respectively, e.g., at least twice the brain dimension in the beam direction. For the 300 kHz frequency in the human case, a 300 μs simulation duration was used to compensate the low attenuation. Longer simulation durations did not yield significant change of the pressure fields and can led to longer, more difficult computations. The large attenuation of a wave reflected more than twice due to the combination of absorption and diffraction is believed to be the main cause, as the standing waves decrease rapidly upon reflection at the brain-skull interface.

Method 100 can further include calculating 180 the formation of standing waves in proximity to the tissue structure of interest. In one embodiment, standing waves can be calculated 180 using a high spatial filter on the maximum pressure field to isolate spatial modulation due to constructive and destructive interferences of the waves. FIGS. 8A and 8B illustrate calculations 180 of standing waves. FIG. 8A illustrates where a plane wave reflected on a 45° interface, the interference of the incident and reflected waves yield a stationary wave with a typical spatial modulation which is detected by the filter. FIG. 8B illustrates that in the case of a focused wave, the high spatial frequencies occurring around the focal spot can yield small artifacts in the detection. A Hilbert transform can be used to obtain the slow variation or envelope of the modulations. The resulting field represents an estimation of the amplitude of the standing wave component. In some embodiments, artifacts were visible in the focal spot, between the lobes, where high spatial frequencies are naturally present, as illustrated in FIG. 8A and 8B.

In some embodiments, peak attenuation and peak displacements compared to water can be determined and a quantification of the targeting parameters can be determined. The Percent-of-Target-Reached parameter (PTR) is the percent volume of the target above the half pressure threshold. A 100% PTR indicates a case where the beam encompasses all the volume of the targeted structure with a pressure higher than half of the peak pressure. Due to the focal spot dimensions expected with a spherical transducer, where a 100% PTR case in a single sonication location is not possible, multiple sonications can be utilized to increase the volume reached inside the target. Diffusion effects can allow, in the case of a BBB application, drugs to be delivered over a larger volume ratio than the PTR itself. The Percent-of-Beam-Overlapping-Target parameter (PBOT) is the volume fraction of the beam above the half pressure of the peak pressure that is inside the target. A 100% PBOT indicates a case where the beam did not reach any collateral structure. Values for targeting without the skull can also be determined to, for example, better assess the skull effects. Standing waves effects can be determined by estimating the ratio of their maximum amplitude to the peak pressure with skull (Standing Wave Maximum Amplitude to Peak Ratio) as well as the percent volume of the brain where their amplitude is higher than 5% of the peak pressure with the skull (Percent of Standing Wave Volume in Brain).

In embodiments utilizing method 200 in BBB applications, avoiding complete formation of standing waves could limit the enhanced cavitational effect due to microbubbles trapped in antinodes. Additionally, standing waves between the transducer and the skull can lead to inconsistently transmitted pressures depending on whether positive or destructive interferences occur.

In one embodiment, fast periodic linear chirp waveforms can be used to limit the standing wave effect. The use of chirps is one technique to reduce the standing wave pattern: by varying the frequency of the signal in time, a time-dependent phase difference between the incident and reflected waves appears. Another technique proposes to use random frequency modulation. In some embodiments, although the amplitude of the reflected wave is not modified, the constructive and destructive interferences of the waves change position over time. A standing wave interference pattern can be present but will change location between time processes. Considering a longer time scale, the effects of constructive and destructive interference can compensate each other and the summation of both waves can appear to be incoherent.

In one embodiment, a fast linear chirp with a period of 23 μs between 450 and 550 kHz was chosen. FIG. 9 illustrates the maximum pressure field while targeting the hippocampus through the human skull using a linear chirp at 450-550 kHz. The dashed white line denotes the contour of the hippocampus.

FIG. 10A illustrates pressure fields with a 500 kHz monochromatic beam and with a 450-550 kHz linear chirp (ratio to the peak pressure in water). As illustrated, interferences patterns are largely reduced by the use of the chirp. The white arrow in FIG. 10A indicates the constructive interferences every 38.5 mm due to the duration of the chirp used. FIG. 1013 illustrates the standing wave amplitude with a 500 kHz monochromatic beam and with a 450-550 kHz linear chirp (in percent of the peak pressure through the skull at a given frequency). The maximum is found over the entire duration of the chirp, e.g., 23 μs. The white contour denotes the volume where the modulation amplitude is higher than 5% of the peak pressure through the skull. As illustrated, this volume decreases from 0.87% to 0.17% of the brain volume when using a chirp. As illustrated, a short period of 23 μs can lead to constant phase differences with travel path differences equal to 38.5 mm, where attenuation can limit the reflected wave amplitude. Such a configuration requires only a short averaging time to suppress the standing wave. In some embodiments, biological or physical effects with a time scale larger than 23 μs will be insensitive to standing waves.

Method 100 further includes utilizing the determined acoustic properties to align 190 the simulated transducer with the tissue structure of interest, e.g., the hippocampus, putamen, caudate, or other tissue structure, such that the tissue structure is targeted. In an embodiment involving human and primate brains, the simulated transducer beam axis can traverse the parietal bone, close to the lambdoid suture, as illustrated in FIG. 4, for both the primate and human skulls cases for targeting the hippocampus and the caudate. The putamen targeting in the human skull can cross the occipital bone below and next to the lambdoid suture. In the primate case, the putamen can be more difficult to target due to its natural orientation and the fact that the beam has to traverse the frontal bone at the top of the skull, close to the coronal suture. The rhesus monkey skull has a large occipital protuberance and brow ridge, as illustrated in FIG. 4, that can cause high aberrations and attenuation on the ultrasound beam if the beam crosses its path.

In one embodiment involving a human brain, the hippocampus was selected 110 as the tissue structure of interest and the transducer was selected 160 to have a 500 kHz frequency. The peak pressure attenuation through the skull was determined 170 to be around 76% compared to that in water, the peak position was determined 170 to be displaced by approximately 13 mm (13 mm along the beam axis and 1.1 mm in the transverse plane). The Percent-of-Target-Reached parameter was calculated to be around 11% (14% without the skull) and the Percent-of-Beam-Overlapping-Target parameter was calculated to be around 69% (76% without the skull). In embodiments utilizing method 100 in BBB opening application, diffusion of the compounds can be utilized to reach a larger volume of the targeted structure and additional sonications can also be used to increase the BBB opened volume.

FIG. 11 illustrates the maximum pressure field at 500 kHz while targeting the hippocampus through a human skull. The dashed white line in FIG. 11 denotes the contour of the hippocampus. As illustrated in FIG. 11, some standing wave interference patterns are visible both between the skull and the transducer. As illustrated in FIGS. 10A and 10B, some standing wave interference patterns are visible inside the brain close to the skull interface. Standing waves are concentrated close to the skull interface where both incident and reflected waves have high amplitudes, which slowly decrease out of this point. The peak amplitude of standing waves was determined to be 19% of the peak pressure through the skull. Only 0.87% of the brain volume was found to have a significant standing wave component higher than 5% of the maximum peak pressure through the skull.

In an embodiment involving human brains where the hippocampus was selected 110 as the tissue structure of interest, the transducer was selected 160 to operate at 300, 500 and 700 kHz frequencies were investigated using the parameters shown in Table 2, above. The acoustic properties determined 170 in the simulations are summarized in Table 5.

TABLE 5
Acoustic properties for the hippocampus in
the human skull for different frequencies.

| | 300 kHz | 500 kHz | 700 kHz |
|---|---|---|---|
| Peak attenuation | 42% | 76% | 88% |
| Peak displacement | 8.3 mm axial + | 13 mm axial + | 28 mm axial + |
| | 1.8 mm lateral | 1.1 mm lateral | 3.8 mm lateral |
| Percent of Target | 13% (21% | 11% (14% | 10% (12% |
| Reached | without | without | without |
| | the skull) | the skull) | the skull) |
| Percent of Beam | 91% (85% | 69% (76% | 56% (84% |
| Reaching | without | without | without |
| Target | the skull) | the skull) | the skull) |
| Standing waves | 26% | 19% | 14% |
| maximum amplitude | | | |
| Standing Waves | 3.7% | 0.87% | 0.23% |
| volume | | | |

FIG. 12A illustrates the pressure fields at 300, 500 and 700 kHz (in percent of the peak pressure in water for the same frequency). The dashed line denotes the contour of the hippocampus and the white arrow indicates the secondary peak at the 700 kHz frequency. FIG. 12B illustrates the corresponding standing wave amplitudes for the same frequencies shown in FIG. 12A (in percent of the peak pressure through the skull at a given frequency). The white contour denotes the volume where the modulation amplitude is higher than 5% of the peak pressure through the skull. As illustrated, this volume decreases with the frequency from 3.7% (at 300 kHz) to 0.23% (at 700 kHz) of the brain volume. The white asterisk indicates the primary brain-skull interface where most of the standing wave effects are concentrated.

At 700 kHz, the secondary peak shown in FIG. 12A, visible already at 500 kHz in FIG. 11, had higher amplitude than the main lobe leading to very large peak displacement (28 mm axially). The volume of standing waves (where spatial modulation amplitude is higher than 5% of the peak pressure through skull at the same frequency) is further reduced with frequency. Because of the very low attenuation in the 300 kHz case, the steady state was assumed to be at 300 μs instead of 200 μis.

In an embodiment where the putamen was selected 110, the peak pressure was determined 170 to be attenuated by around 85% compared to that in water, the peak position was displaced by approximately 3.9 mm (3.5 mm along the beam axis and 1.8 mm in the transverse plane). FIG. 13 illustrates the maximum pressure field at 500 kHz while targeting the putamen through the human skull. The dashed white line denotes the contour of the putamen. The Percent-of-Target-Reached parameter was estimated to be around 22% and the Percent-of-Beam-Overlapping-Target parameter around 28%.

The peak amplitude of standing waves was calculated 180 to be 17% of the peak pressure through the skull and 1.5% of the brain was determined 180 to have a standing wave component higher than 5% of the maximum peak pressure with the skull, this volume was concentrated around the beam-skull interface.

FIG. 14 illustrates the maximum pressure field at 500 kHz were the caudate was the tissue structure selected 110 for targeting. The dashed white line denotes the contour of the caudate.

In an embodiment involving human brain structures where the hippocampus was selected 110, a linear chirp was used to reduce the standing waves influence. Standing waves interference patterns can nearly be eliminated from the maximum pressure field, as illustrated in FIGS. 10A and 10B. The standing wave maximum amplitude was calculated 180 to be 12% of the peak pressure through the skull. Only 0.17% of the brain volume was calculated 180 to have a significant standing wave component (higher than 5% of the maximum peak pressure through the skull). The reduction is thus significant compared to the monochromatic case (5× reduction). In this embodiment, the maximum standing wave amplitude is localized close to the skull interface, where the time difference between the incident and reflected waves is small, leading to a small frequency difference with interferences comparable to the monochromatic beam. This can result in a smaller reduction of the peak amplitude of standing waves compared to the monochromatic case is not as significant (e.g., a 1.5× reduction).

In one embodiment involving a primate brain, the hippocampus was selected 110 as the tissue structure of interest and the transducer was selected 160 to have an 800-kHz frequency. FIG. 15 illustrates the maximum pressure field at 800 kHz obtained with targeting the hippocampus through the primate skull. The white dashed line denotes the hippocampus contour. As illustrated, part of the beam was absorbed when passing through the occipital protuberance. Attenuation through the primate skull determined 170 to be around 80% compared to water and the peak position was determined 170 by approximately 6.4 mm (6.2 mm along the beam axis and 1.6 mm in the transverse plane). The Percent-of-Target-Reached parameter was calculated to be around 18% (17% without the skull) and Percent-of-Beam-Overlapping-Target parameter (Volume of pressure above 50% of peak that is inside target) was calculated to be around 31% (72% without the skull). In an application utilizing method 100 in a BBB application, most of the BBB volume opened would be in collateral structure surrounding the hippocampus.

FIG. 16 illustrates the maximum pressure field at 800 kHz obtained with targeting the putamen through the primate skull. The white dashed line denotes the putamen contour. As illustrated in FIG. 16, compared to the targeting of the hippocampus, the attenuation increases from 80% to 93% with strong secondary peaks. FIG. 17 illustrates the maximum pressure field at 800 kHz obtained with targeting the caudate through the primate skull. The white dashed line denotes the caudate contour. As illustrated, the large incidence angle due to the primate skull curvature leads to a strong reflected wave and an attenuation of 97%.

In this embodiment involving primate brains, standing waves were determined to be concentrated close to the skull interface. FIGS. 18A and 18B illustrate the standing wave amplitude at 600, 800 and 1000 kHz (in percent of the peak pressure through the skull at a given frequency). The white contour denotes the volume where the modulation amplitude is higher than 5% of the peak pressure through the skull. As illustrated, this volume decreases with the frequency from 3 to 0.64% of the brain volume. The maximum amplitude of standing waves was calculated 180 to be 13% of the peak pressure through the skull. A volume of 1.4% of the brain was calculated 180 to have a standing wave component higher than 5% of the maximum peak pressure within the skull. Some artifacts can be visible due to the high spatial frequency of the field close to the focus.

In an embodiment involving primate skulls where the hippocampus was selected 110 as the tissue structure of interest, the transducer was selected 160 to operate at 600 kHz and 800 kHz frequencies were investigated using the parameters shown in Table 2. The acoustic properties determined 170 in the simulations are summarized in Table 6.

TABLE 6
Targeting features for the hippocampus in
the monkey skull for different frequencies.

| | 600 kHz | 800 kHz |
|---|---|---|
| Peak attenuation | 68% | 80% |
| Peak displacement | 5.8 mm axial + | 6.2 mm axial + |
| | 1.4 mm lateral | 1.3 mm lateral |
| Percent-of-Target-Reached | 24% (29% | 18% (17% |
| | without | without |
| | the skull) | the skull) |
| Percent-of-Beam-Overlapping- | 36% (73% | 31% (76% |
| Target | without | without |
| | the skull) | the skull) |
| Standing waves maximum | 22% | 13% |
| amplitude | | |
| Standing waves volume | 3% | 1.4% |

FIG. 19A illustrates a system 300 used in an exemplary embodiment to perform in vivo BBB opening in an anesthetized live monkey (e.g., a Macaca Mulatta). The transducer 340 was positioned with a stereotactic unit 350 in the dorsal part of the primate's head 1900 in order to target the putamen at 500 kHz. In this embodiment, pressures predicted by system 300 were sufficient to induce BBB opening in monkeys. Pressures of 0.45 MPa and 0.6 MPa were sufficient to induce BBB opening in two separate regions through the intact skull and scalp as evidenced by MR imaging (3.0T Intera, Philips) using gadolinium to indicate the region where the BBB was opened. FIG. 19B illustrates the regions of opening detected on the same coronal plane with the solid circle illustrating the opening produced by the 0.45 MPa sonication and dashed circle illustrating the larger opening produced by the 0.6 MPa sonication. FIGS. 19C and 19D illustrate two separate sagittal planes, as a contrast enhancement. A shift predicted by system 300 occurred, which placed the beam a few millimeters anterior to the region targeted.

The foregoing merely illustrates the principles of the disclosed subject matter. Various modifications and alterations to the described embodiments will be apparent to those skilled in the art in view of the teachings herein. Features of existing methods can be seamlessly integrated into the methods of the exemplary embodiments of the disclosed subject matter or a similar method. It will thus be appreciated that those skilled in the art will be able to devise numerous methods and systems which, although not explicitly shown or described herein, embody the principles of the disclosed subject matter and are thus within its spirit and scope.

We claim:

1. A method for targeting a tissue structure using corresponding tissue structure image data, comprising:
receiving the tissue structure image data into a targeting simulator;
determining one or more acoustic properties of the tissue structure from the corresponding tissue structure image data; and
utilizing the determined one or more acoustic properties to align a simulated transducer with the tissue structure such that the tissue structure is targeted.

2. The method of claim 1, further comprising acquiring the tissue structure image data.

3. The method of claim 2, wherein the acquiring comprises acquiring a CT scan of at least the tissue structure.

4. The method of claim 2, wherein the acquiring comprises acquiring an MRI of at least the tissue structure.

5. The method of claim 1, further comprising aligning the tissue structure image data with an atlas of a body structure encompassing the tissue structure.

6. The method of claim 1, further comprising selecting parameters of the simulated transducer such that a focal region of an ultrasound wave generated by the simulated transducer targets the tissue structure.

7. The method of claim 1, wherein the simulated transducer generates an ultrasound wave, and further comprising calculating one or more standing wave properties of the ultrasound wave in proximity to the tissue structure.

8. The method of claim 1, wherein the determining comprises determining a pressure waveform of an ultrasound wave moving from the simulated transducer to the tissue structure.

9. A method for applying ultrasound to a tissue structure using corresponding tissue structure image data, comprising:
receiving the tissue structure image data into a targeting simulator;
determining one or more acoustic properties of the tissue structure from the corresponding tissue structure image data; and
utilizing the determined one or more acoustic properties to align a simulated transducer with the tissue structure such that the tissue structure is targeted;
utilizing the alignment of the simulated transducer to align a real transducer with the tissue structure; and
applying ultrasound to the tissue structure using the real transducer.

10. The method of claim 9, further comprising monitoring the application of ultrasound to the tissue structure.

11. The method of claim 9, wherein applying comprises applying ultrasound utilizing transducer parameters effective to open the tissue structure.

12. The method of claim 9, wherein applying comprises applying ultrasound utilizing transducer parameters effective to reduce formation of standing waves in proximity to the tissue structure.

13. The method of claim 11, wherein the tissue structure comprises a brain structure and wherein opening the tissue structure comprises opening a blood brain barrier.

14. A system for targeting a tissue structure using corresponding tissue structure image data, comprising:
a targeting simulator comprising a processing unit operatively connected to a memory unit and an input unit, wherein the memory unit contains program instructions operable, when executed by the processing unit, to:
receive the tissue structure image data;
determine one or more acoustic properties of the tissue structure from the corresponding tissue structure image data; and
utilize the determined one or more acoustic properties to align a simulated transducer with the tissue structure such that the tissue structure is targeted.

15. The system of claim 14, wherein the program instructions are further operable, when executed by the processing unit, to:
align the tissue structure image data with an atlas of a body structure encompassing the tissue structure;
receive parameters of the simulated transducer selected such that a focal region of an ultrasound wave generated by the simulated transducer targets the tissue structure; and
calculate one or more standing wave properties of the ultrasound wave in proximity to the tissue structure.

16. The system of claim 14, further comprising one or more image acquisition devices for acquiring the tissue structure image data.

17. A system for applying ultrasound to a tissue structure, comprising:
an ultrasound transducer; and
a targeting simulator comprising a processing unit operatively connected to a memory unit and an input unit, wherein the memory unit contains program instructions operable, when executed by the processing unit, to:
receive the tissue structure image data;
determine one or more acoustic properties of the tissue structure from the corresponding tissue structure image data; and
utilize the determined one or more acoustic properties to align a simulated transducer with the tissue structure such that the tissue structure is targeted.

18. The system of claim 17, wherein the program instructions are further operable, when executed by the processing unit, to:
monitor an application of ultrasound to the tissue structure.
